(19) United States
(12) Patent Application Publication
(10) Pub. No.: US 2001/0012636 A1
(43) Pub. Date: Aug. 9, 2001

(54) Chewing gum with pH indicator

(75) Inventors: Zion Azar, Shoham (IL); Pinchas Shalev, Kfar Saba (IL)

Correspondence Address: BIRCH STEWART KOLASCH & BIRCH, PO BOX 747, FALLS CHURCH, VA, 22040-0747, US

(73) Assignee: Radiancy Inc.

(21) Appl. No.: 09/818,771

(22) Filed: Mar. 28, 2001

Related U.S. Application Data
(62) Division of application No. 09/377,498, filed on Aug. 20, 1999.

(51) Int. Cl. G01N 33/52; A61K 6/00

(52) U.S. Cl. 436/163; 422/61; 424/9.7; 424/10.32; 424/410; 424/415; 426/87; 426/106; 426/132

(57) ABSTRACT

A pH sensitive chewing gum kit is provided. The kit may contain chewing gum incorporating a pH sensitive substance. When chewed, the gum may change it's color in response to the oral fluids or salivary pH level. The chewing gum kit may also contain chewing gum with or without artificial sweeteners and one or more pH sensitive members including at least one pH sensitive substance incorporated in at least one portion thereof. When a pH sensitive member is put in contact with oral fluids or saliva, before, during or after chewing the gum, the color of the pH sensitive substance may change to a color which depends on the oral fluids pH value. This color may indicate whether the gum still needs to be chewed in order to achieve a desired oral fluids pH level. The kit may also include a reference color scale for assisting the interpretation of the color changes of the gum or of the pH sensitive members by the user to inform the user whether the gum still needs to be chewed and, optionally, for informing the user of the approximate remaining chewing time required to bring the oral fluids pH level to a desired value. Preferably, the desired value is higher (more basic) than a critical pH level of 5.3.

### FIELD OF THE INVENTION

The present invention relates to the field of oral hygiene. More particularly it relates to a chewing gum provided with a pH indicator which allows checking of the oral pH levels.

### BACKGROUND OF THE INVENTION

It is known that dental caries is caused by interaction between oral bacteria, their access to fermentable carbohydrates and vulnerable parts of the tooth. The widely known Stephen's graph (FIG. 1) shows the steep drop in plaque pH after a glucose rinse. The drop in pH is the result of fermentation of carbohydrates by some bacteria. A major factor contributing to plaque acidification is the production of lactic acid and other acids as a bacterial carbohydrate fermentation byproduct.

The gradual return of the pH is the result of buffers present in plaque and saliva. Provided the pH does not drop below 5.3 the dental enamel remains intact, but below this critical level, crystals of apatite dissolve. Fortunately, both plaque and saliva are saturated with calcium and phosphate ions, so that if the pH returns fairly rapidly above the 5.3 level, ions will go back into the enamel and recrystallize. This process of remineralization takes longer in an acid environment, but is rapid if the fluid next to the enamel is neutral or even alkali. Caries is therefore uncommon in those parts of the mouth near the outflow of salivary glands, like the lower incisors, where the teeth are constantly bathed with the buffers and concentrated calcium ions of saliva. If the total outflow of saliva can be increased, there is a greater chance of protection of all the teeth in the arch. Some foods, like cheese, stimulate the flow of saliva. It was observed that cheese after a meal was not only a pleasant way to end it, but also a particularly good caries inhibitor. Sugar is also a good stimulator of saliva but of course also provides most favored nutrients for plaque bacteria. However, sugar substitutes, while of no use to bacteria, are also effective saliva stimulants, as is the mere act of chewing, so chewing a gum which is artificially sweetened makes biological sense, and indeed has a role in arresting caries.

Thus, several types of artificially sweetened chewing gum are commercially available and are often recommended by oral hygiene professionals for use after consuming a meal or a snack as an effective means to restore oral pH to favorable levels. Such artificially sweetened or “sugar free” chewing gum, may include xylitol, sorbitol, mannitol, maltitol syrup as sweetening agents or may include other artificial non-sugar sweeteners such as aspartame, saccharine and cyclamate or the like, substituting for glucose. The advantage of using sweeteners like xylitol or sorbitol or the like, or other artificial sweeteners is that they are not efficiently metabolized by plaque bacteria and therefore do not efficiently contribute to plaque acidification. For example, a sorbitol sweetened chewing gum under the trademark “Orbit® sugarfree” is commercially available from Wm. Wrigley Jr. Company, USA. Other varieties of chewing gum directed to fighting plaque and caries may include agents like sodium bicarbonate (also known as baking soda) which may neutralize plaque acids and help restore favorable oral pH. For example, Duane Reade, U.S.A., commercially distributes a dental gum containing baking soda.

While chewing gum seems as an efficient oral hygiene measure in restoring favorable pH levels in the oral cavity, it is difficult to tell exactly how long one must chew a gum in order to restore the pH to a value above the pH 5.3 level.

One possible method used to monitor the oral pH is the use of electronic pH meter devices. While desktop pH devices may be suitable for oral and dental research purposes, they are too large, cumbersome and expensive for routine daily use. Small, hand held pH meters are now commercially available which are small enough and portable and may be used for determining oral pH. However, such hand held pH meters have disadvantages which prevent their routine use for checking the pH restoring effects of chewing dental gum on a regular basis. Among such disadvantages is the cost of hand held pH meters, the complexity of their use, the need for their calibration, and the fact that they may have to be repeatedly inserted into the oral cavity, raising esthetic and hygenic problems.

### SUMMARY OF THE INVENTION

An aspect of the present invention is the providing of a pH indicating chewing gum. The chewing gum includes a pH indicator which indicates the oral pH level during or after the chewing, and allows checking of the pH levels in one's saliva or oral fluids.

Another aspect of the present invention is the providing of a dental gum kit which includes a dental chewing gum and one or more pH indicating means. The pH indicating means may be a pH indicating member such as a pH test strip or toothpick like member or the like which has at least one part thereof incorporating one or more pH indicator substances.

The pH indicating member may also be the chewing gum wrapper or a part thereof.

A further aspect of the invention is that, preferably, non-toxic and/or edible pH indicator substances are used in implementing the various embodiments of the invention either by incorporation thereof within the chewing gum or by including them within the pH indicating means of the various embodiments of the invention.

Another aspect of the present invention is that a reference color scale may be included in the dental gum kit or supplied with the pH indicating chewing gum for assisting the user to interpret the results of the pH check performed using the dental gum kit or the pH indicating chewing gum.

There is therefor provided, in accordance with a preferred embodiment of the present invention, a chewing gum for checking the pH of oral fluids. The chewing gum includes a gum base and at least one pH sensitive substance dispersed in the gum base.

Furthermore, in accordance with another preferred embodiment of the present invention, the pH sensitive substance is capable of responding to the pH level of the oral fluids by undergoing a visibly detectable color change.

Furthermore, in accordance with another preferred embodiment of the present invention, the chewing gum further includes at least one sweetening substance.

Furthermore, in accordance with another preferred embodiment of the present invention, the pH sensitive substance is adapted to responds to pH changes in the range of 4.0-6.0 by visibly changing its color.

Furthermore, in accordance with another preferred embodiment of the present invention, the visibly detectable color change is a visibly detectable change from a colorless to a colored form, a visibly detectable change from a colored form to a colorless form or a visibly detectable change from a first color to a second color.

Furthermore, in accordance with another preferred embodiment of the present invention, the pH sensitive substance includes one or more anthocyanin dyes obtained from red cabbage, and the visibly detectable color change includes a change in the color of the chewing gum from a red color at a pH of approximately 4 to a blue color at a pH of approximately 6.

Furthermore, in accordance with another preferred embodiment of the present invention, the pH sensitive substance is a non-toxic pH indicator dye.

Furthermore, in accordance with another preferred embodiment of the present invention, the pH sensitive substance is an anthocyanin pH indicator dye.

Furthermore, in accordance with another preferred embodiment of the present invention, the pH sensitive substance is an edible anthocyanin preparation obtained from red cabbage.

Furthermore, in accordance with another preferred embodiment of the present invention, the chewing gum further includes at least one substance selected from an emulsifier, an antioxidant, a stabilizer and a flavoring substance.

There is further provided, in accordance with a preferred embodiment of the present invention, a dental gum kit including at least one piece of chewing gum including a gum base and at least one pH sensitive substance dispersed in the gum base, and a reference color scale for assisting a user of the gum kit to interpret the results of a pH check performed by chewing the chewing gum.

Furthermore, in accordance with another preferred embodiment of the present invention, the pH sensitive substance is capable of responding to the pH level of the oral fluids by undergoing a visibly detectable color change.

There is still further provided, in accordance with a preferred embodiment of the present invention, a gum kit. The gum kit includes at least one piece of chewing gum and at least one pH sensitive member for checking the pH level of oral fluids.

Furthermore, in accordance with another preferred embodiment of the present invention, the pH sensitive member is an elongated strip-like member having at least one portion including at least one pH sensitive substance capable of responding to the pH of oral fluids by undergoing a visibly detectable color change.

Furthermore, in accordance with another preferred embodiment of the present invention, the pH sensitive member includes an elongated absorbent member and the at least one portion of the elongated absorbent member is impregnated with at least one pH sensitive substance.

Furthermore, in accordance with another preferred embodiment of the present invention, the at least one pH sensitive member is made of a material selected from wood, paper, and a porous absorbent synthetic polymer.

Furthermore, in accordance with another preferred embodiment of the present invention, the at least one pH sensitive member includes an applicator and at least one piece of absorbent material impregnated with the at least one pH sensitive substance.

Furthermore, in accordance with another preferred embodiment of the present invention, the applicator is a plastic applicator and the at least one piece of absorbent material is made of a material selected from wood, paper, and a porous absorbent synthetic polymer.

Furthermore, in accordance with another preferred embodiment of the present invention, the at least one piece of chewing gum is disposed within a gum wrapper, and the at least one pH sensitive member includes the gum wrapper or a portion thereof.

Furthermore, in accordance with another preferred embodiment of the present invention, the gum wrapper includes a reference color scale for assisting a user of the gum kit to interpret the results of an oral pH check performed using the gum wrapper or the portion thereof.

Furthermore, in accordance with another preferred embodiment of the present invention, the gum wrapper includes a plurality of portions. Each of the portions is usable for checking the pH of oral fluids.

Furthermore, in accordance with another preferred embodiment of the present invention, at least a first portion of the plurality of portions is adapted to undergo a visibly detectable color change within a first pH range and at least a second portion of the plurality of portions is adapted to undergo a visibly detectable color change within a second pH range.

Furthermore, in accordance with another preferred embodiment of the present invention, the first pH range and the second pH range at least partially overlap.

Furthermore, in accordance with another preferred embodiment of the present invention, the first pH range and the second pH range do not overlap.

Furthermore, in accordance with another preferred embodiment of the present invention, the plurality of portions are separable portions.

Furthermore, in accordance with another preferred embodiment of the present invention, the separable portions are separated from each other by tearing the wrapper along at least one tearing guide line.

Furthermore, in accordance with another preferred embodiment of the present invention, the at least one tearing guide line is a fold line or a perforated guide line.

Furthermore, in accordance with another preferred embodiment of the present invention, the at least one pH sensitive member is a toothpick-like member having at least a portion thereof including the at least one pH sensitive substance.

Furthermore, in accordance with another preferred embodiment of the present invention, the toothpick-like member is further adapted to be used for performing oral hygiene operations such as tooth picking and plaque removing.

Furthermore, in accordance with another preferred embodiment of the present invention, the gum kit further includes a reference color scale for assisting a user of the gum kit to interpret the results of a pH check performed using the pH sensitive member.

Furthermore, in accordance with another preferred embodiment of the present invention, the gum kit further includes a container for holding the at least one piece of chewing gum and the at least one pH sensitive member, and the reference color scale is disposed within the container.

Furthermore, in accordance with another preferred embodiment of the present invention, the container includes a gum kit wrapper, and the reference color scale is disposed within the gum kit wrapper or printed thereupon.

Furthermore, in accordance with another preferred embodiment of the present invention, the gum kit further includes a container for holding the at least one piece of chewing gum and the at least one pH sensitive member, and the reference color scale is attached to or printed on the container.

There is further provided, in accordance with a preferred embodiment of the present invention, a method for preparing chewing gum for checking the pH of oral fluids. The method includes the steps of providing a chewable gum base, and dispersing at least one pH sensitive substance in the gum base. The at least one pH sensitive substance is capable of responding to the pH level of the oral fluids by undergoing a visibly detectable color change.

Furthermore, in accordance with another preferred embodiment of the present invention, the method further includes the step of dispersing at least one sweetening substance in the gum base.

Furthermore, in accordance with another preferred embodiment of the present invention, the sweetening substance is selected from the group including sorbitol, xylitol, mannitol, maltitol syrup aspartame, sacharine, cyclamate and any combination thereof.

Furthermore, in accordance with another preferred embodiment of the present invention, the at least one pH sensitive substance responds to pH changes in the range of 4.0-6.0 by undergoing a visibly detectable color change.

Furthermore, in accordance with another preferred embodiment of the present invention, the visibly detectable color change is selected from a visibly detectable change from a colorless to a colored form, a visibly detectable change from a colored form to a colorless form and a visibly detectable change from a first color to a second color.

Furthermore, in accordance with another preferred embodiment of the present invention, the at least one pH sensitive substance includes one or more anthocyanin dyes obtained from red cabbage, and the visibly detectable color change includes a change in the color of the chewing gum from a red color at a pH of approximately 4 to a blue color at a pH of approximately 6.

Furthermore, in accordance with another preferred embodiment of the present invention, the at least one pH sensitive substance is a non-toxic pH indicator dye.

Furthermore, in accordance with another preferred embodiment of the present invention, the at least one pH sensitive substance is an anthocyanin pH indicator dye.

Furthermore, in accordance with another preferred embodiment of the present invention, the at least one pH sensitive substance is an edible anthocyanin preparation obtained from red cabbage.

Furthermore, in accordance with another preferred embodiment of the present invention, the method further includes the step of adding to the gum base at least one substance selected from a softening substance, a flavoring substance, an antioxidant substance and an emulsifier substance.

There is further provided, in accordance with a preferred embodiment of the present invention, a method of preparing a gum kit for performing oral pH checks. The method includes the steps of providing at least one piece of chewing gum, and providing at least one pH sensitive member adapted for checking the pH level of oral fluids.

Furthermore, in accordance with another preferred embodiment of the present invention, the at least one pH sensitive member is adapted for checking the pH level of oral fluids at a time selected from before the chewing of the chewing gum, during the chewing of the chewing gum and after the chewing of the chewing gum.

There is further provided, in accordance with a preferred embodiment of the present invention, a method of preparing a gum kit for checking oral fluids pH level. The method includes the steps of providing at least one piece of chewing gum, and providing means for checking the oral fluids pH level.

Furthermore, in accordance with another preferred embodiment of the present invention, the means for checking the oral pH level is adapted for checking the oral fluids pH level at a time selected from before the chewing of the chewing gum, during the chewing of the chewing gum and after the chewing of the chewing gum.

Furthermore, in accordance with another preferred embodiment of the present invention, the means for checking the oral pH level includes at least one pH sensitive member capable of responding to the pH of oral fluids by undergoing a visibly detectable color change.

Furthermore, in accordance with another preferred embodiment of the present invention, the means for checking the oral pH level includes at least one piece of pH sensitive gum capable of responding to the pH level of oral fluids by undergoing a visibly detectable color change.

Furthermore, in accordance with another preferred embodiment of the present invention, the method further includes the step of providing a reference color scale for assisting a user of the chewing gum to interpret changes in the color of the means for checking the oral pH level. The changes in color result from chewing the gum.

There is further provided, in accordance with a preferred embodiment of the present invention, a chewing gum wrapper including a wrapping sheet. At least a portion of the wrapping sheet includes at least one pH sensitive substance.

Furthermore, in accordance with another preferred embodiment of the present invention, the at least one pH sensitive substance is sensitive to pH level changes, and is capable, after being wet by oral fluids, of responding to the pH level of the oral fluids by undergoing a visibly detectable color change.

Finally, in accordance with another preferred embodiment of the present invention, the oral fluids are selected from saliva, a liquid introduced into the oral cavity, a liquid containing food particles dispersed therein, and any combination thereof.

### BRIEF DESCRIPTION OF THE FIGURES

In order to better understand the present invention, and appreciate its practical applications, the following Figures are provided and referenced hereinafter. It should be noted that the Figures are given as examples only and in no way limit the scope of the invention as defined in the appending Claims. Like components are denoted by like reference numerals.

FIG. 1 (prior art) is a schematic Stephen's graph, illustrating the relation between plaque pH and time, before and after a glucose mouth rinse, and the effect of chewing an artificially sweetened chewing gum on plaque pH;

FIG. 2 illustrates a wrapped chewing gum with pH indicator included in the wrapper, in accordance with a preferred embodiment of the present invention;

FIG. 3A is a schematic diagram illustrating the unfolded chewing gum wrapper with pH indicator of FIG. 2, in accordance with a preferred embodiment of the present invention;

FIG. 3B is a schematic diagram illustrating a gum wrapper which may be longitudinally separated into a plurality of pH sensitive members, in accordance with another preferred embodiment of the present invention;

FIG. 4 is a schematic diagram of a chewing gum wrapper having a plurality of portions of paper impregnated with a pH sensitive indicator substance attached thereto, in accordance with a preferred embodiment of the present invention;

FIGS. 5A-5F are schematic diagrams illustrating various forms of pH sensitive members, usable in dental gum kits for checking the pH level of oral fluids, in accordance with additional preferred embodiments of the present invention;

FIG. 5G is a schematic diagram illustrating a reference color scale adapted for use with the pH sensitive member of FIG. 5F;

FIG. 6 is a schematic isometric view of a gum kit including a color reference scale positioned on the wrapper of the kit;

FIG. 7 is a schematic isometric view of a gum wrapper including a reference color scale useful for indicating the recommended time of chewing a gum based on the results of an oral fluids pH check, in accordance with a preferred embodiment of the present invention; and

FIG. 8 which is a schematic diagram of a dental gum kit, in accordance with yet another preferred embodiment of the present invention.

### DETAILED DESCRIPTION OF THE INVENTION

It is noted that the expressions “pH sensitive substance” and “pH indicator substance” are interchangeably used throughout the present application and refer to a substance that is capable of changing its color in response to pH changes.

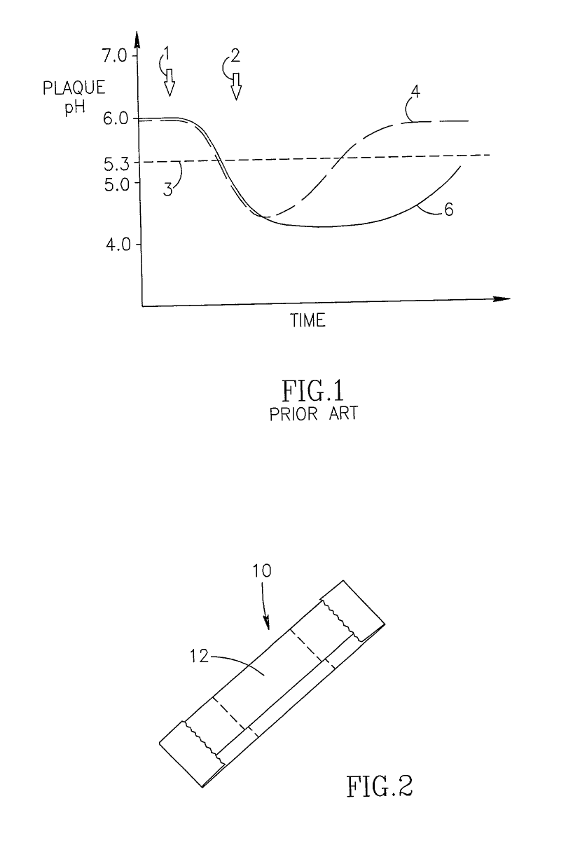

Reference is made to FIG. 1 (prior art) which schematically illustrates Stephen's graph, plotting the relation between plaque pH and time, before and after a glucose mouth rinse and the effect of chewing an artificially sweetened chewing gum on plaque pH.

The vertical axis of the graph represents plaque pH and the horizontal axis represents time. The curve 6 represents the time course of plaque pH after a glucose rinse and the curve 4 represents the time course of plaque pH after a glucose rinse followed by chewing an artificially sweetened chewing gum. The dashed horizontal line 3 represents the critical pH level of pH=5.3. Turning to curve 6, the arrow 1 represents the time of application of an oral glucose rinse. After exposing the glucose rinse, the plaque pH level, which is initially at a level of approximately pH 6.0 gradually drops below the critical pH level represented by the dashed line 3. The pH level then may gradually recover due to natural salivation rinsing the oral cavity.

Turning to curve 4, the arrow 1 represents the time of application of an oral glucose rinse and the arrow 2 represents the time of starting the chewing of an artificially sweetened chewing gum. It can be seen that following the cheing of the gum the process of recovery of plaque pH is expedited by chewing an artificially sweetened chewing gum and that the plaque pH of curve 4 crosses the critical pH level represented by the dashed line 3 earlier than in curve 6 reducing the total period in which the teeth are exposed to ph levels more acidic than the critical pH level, which may help in reducing or preventing the development of dental caries.

The present invention seeks to provide, inter alia, an indication of the efficiency of the gum chewing process, by providing a pH indicator incorporated with the chewing gum. It provides a person chewing a gum for the purpose of raising the pH level in his mouth after a meal, a snack or sugar drink, or the consuming of food or drink leading to oral acidification, with an indication as to the progress of the oral fluid or salivary pH level to favorable pH levels. The present invention may also provide the user with an indication or estimate of the approximate time for which the chewing of the gum need to be continued in order to bring the saliva or the oral fluids to a selected desired or pH level which is considered to be favorable for dental health.

Measurement methods of pH levels using pH indicators are well known in the art. A common way to measure pH in a liquid is to expose pH indicator paper to the examined liquid or solution and to compare the color developed on the pH indicator paper with the various colors of a color scale depicting the colors that are produced when the pH indicator paper is exposed to solutions having known reference pH values. Typically, the pH indicator paper is impregnated with one or more pH sensitive substances. The pH sensitive substances used in the preparation of pH indicator paper may be synthetic pH indicator compounds, such as, for example, bromothymol blue, bromocresyl green, phenolphthalein or the like, or naturally occurring substances such as litmus, hematoxilin or the like.

The proposed method of pH measurement of the present invention involves the use of non-toxic synthetic or natural pH indicators. Typically, a pH indicator comprises a weak acid with differently colored acid and conjugate base forms. Many pH indicators can be extracted from plants, or insects such as the indicators alizarin, cochineal, anthocyanin, litmus and the like. It is noted that the mere fact that a pH indicator is extracted from a plant or a living organism does not guarantee that it is not toxic or harmful. For example, some types of litmus, a naturally formed pH indicator extracted from certain types of lichens, are known to be irritants of the eyes, gastrointestinal tract, other mucus membranes, and skin. Care should therefore be taken to implement the present invention with non-toxic and preferably edible pH indicator substances or compounds.

A simple pH indicator is the juice extracted from red cabbage, which is edible and therefore safe for oral use and ingestion. The red cabbage juice or extract contains compounds of the anthocyanin family. The red cabbage extract has the following colors at the following different pH levels, red at pH 2, reddish-purple to purple at pH 4, violet at pH 6, blue at pH 8, blue-green at pH 10 and green at pH 12.

In the relevant salivary or oral fluids' pH range, the red cabbage extract has the following colors, reddish-purple to purple at pH 4, which is about the pH level of acidified plaque reached by the curve 6 of Stephen's graph (of FIG. 1) after the glucose rinse, or after consuming a meal or a snack containing carbohydrates. At about the critical pH level of 5.3 the red cabbage extract has an approximately purple-violet color. At about pH 6 the red cabbage extract has a violet color. At a neutral pH (pH 7), the red cabbage extract has a violet-blue color.

In accordance with one preferred embodiment of the present invention, a pH indicator substance is incorporated within a chewing gum.

Typically, chewing gum is made from a gum base which is a combination of food-grade synthetic and natural ingredients. To this gum base other substances may be added such as, inter alia, natural or artificial sweeteners, softeners, flavoring substances, coloring substances, emulsifiers, antioxidants, and the like.

Gum base may be commercially obtained. For example, a variety of gum base products are commercially available from Gum Base Co. SpA, Milano, Italy.

A gum base kit is commercially available from VERVE, inc. 305 dudley street, Providence, R.I., U.S.A.

It is noted that compositions and methods of preparation of the gum base are well known in the art, are not the subject matter of the present invention and are therefore not disclosed in detail hereinafter.

Thus, in preparing of the pH indicating chewing gum of the present invention, a pH indicator substance is added to a gum base by mixing it with the gum base, preferably, when the gum base is warm. The dispertion or mixing of the pH indicator substance or sdubstances may be performed using any mixing or dispersing methods known in the art. The pH indicator substance may also be premixed with other substances such as, inter alia, natural or artificial sweeteners, softeners, flavoring substances, coloring substances and the like, to form an additive mixture which is then added and blended with the gum base.

Preferably, the pH indicator substance is an edible, non-toxic natural pH indicator substance or a mixture of non-toxic naturally occurring pH indicator substances. However, synthetic, non-toxic pH indicator substances may also be used.

### EXAMPLE 1

The experiment used commercially available chewing gum. A few non-colored, mint flavored ORBIT® chewing gum bars, commercially available commercially available from Wm. Wrigley Jr. Company, USA. Where placed on a plate. A few drops of the food coloring dye, Elderberry Concentrate, article 3266, (Batch No. 21154/21.04.99), commercially obtained from Dr. Marcus GmbH, Germany, were added on the chewing gum pieces to the chewing gum pieces. The plate including the chewing gum pieces and the dye were heated for approximately 15-20 minutes in a microwave oven and then left to cool for a few minutes and then kneaded untill al the dye was absorbed and incorporated in the gum to form a ball like mass of gum having a red color.

A piece of the formed red colored gum was chewed for approximately 2 minutes, at which time the chewed piece of chewing gum has changed it's color from red to blue.

### EXAMPLE 2

The experiment used commercially available chewing gum. Two non-colored, mint flavored ORBIT® chewing gum bars, commercially available commercially available from Wm. Wrigley Jr. Company, USA. Where placed on a plate. A few drops of the food coloring dye, Elderberry Concentrate, article 3266, (Batch No. 21154/21.04.99), commercially obtained from Dr. Marcus GmbH, Germany, were added on the chewing gum pieces to the chewing gum pieces. The plate including the chewing gum pieces and the dye were heated for approximately 15-20 minutes in a microwave oven and then left to cool for a few minutes and then kneaded untill al the dye was absorbed and incorporated in the gum to form a ball like mass of gum having a red color.

The resulting mass of red colord gum was divided into to approximately equal portions. The first portion of red colored gum was immersed for a few minutes in a basic solution prepared by dissolving a half teaspoon of backing soda powder in two glasses of tap water. The first portion of red colored gum changed it's color during the immersion in the baking soda solution from a red color to a blue color.

The second portion of the red colored gum was immersed in a glass of Coca Cola® soft drink, commercially available from Coca Cola. After a few minutes of immersion in the acidic soft drink, the piece of gum was taken out and visually observed. The color of the gum after immersion in the soft drink has changed from red to light red.

It is noted that, while the non-limiting example disclosed hereinabove is based on the use of the above disclosed elderberry concentrate containing pH indicating dye substances, other different anthocyanin based or non-anthocyanin based pH indicating dyes may be used in implementing the pH indicating chewing gum of the present invention. Additionally, while it is preferred to use edible, naturally obtained pH indicator substances in implementing the present invention, synthetic non-natural pH indicating compounds may also be used as long as they are non-toxic, are certified for use in food and provide a suitable visibly detectable color changes when exposed to pH levels in the relevant oral pH range, to usefully indicate the restoration of favorable pH levels.

When the gum containing the pH indicator substance is chewed after consuming a meal, a snack, a soft drink, or any other food or beverage which may change the oral pH level, the chewing of the gum may stimulate the secretion of saliva which contributes to accelerating the restoration of oral pH to favorable pH levels.

The chewing gum containing the pH indicator substance of the present invention enables the user to check the pH levels of oral fluids including saliva. Once the chewing of the gum starts, the pH indicator substance or substances incorporated within the gum is brought in contact with the oral fluids such as but not limited to saliva. The pH indicator substance or substances interact with the oral fluid to change it's color in accordance with the pH level of the oral fluid. Thus, initially, if the acidity of the oral fluid is increased due to carbohydrate related fermentation or to the acidity of a carbonated drink or the like, the gum may change its color due to the change of the pH indicator substance.

Thus, by taking the chewed gum and observing it's color, the person chewing the gum may check the oral pH to determine when the oral pH has reached a favorable pH level. The user may also compare the color of the chewing gum at various times after the commencement of the chewing to standardized reference colors of a reference color scale (not shown) supplied with the gum, as disclosed in detail hereinafter.

It is noted that throughout the present application, the term oral pH refers to the pH level measurable in any fluid within the oral cavity. Such oral fluids include, but are not limited to, saliva, fluids introduced into the oral cavity such as but not limited to beverages, drinks, and fluids or juices contained in a food composition, and any combination thereof. Similarly, throughout the present application the terms “oral pH check”, “checking the oral pH”, and the like, refer to performing a pH check of any oral fluid present in the oral cavity, as disclosed hereinabove.

It will be appreciated by those skilled in the art that, while most of the pH indicator substances disclosed hereinabove respond to a pH change by changing their hue or color from a first color to a second color different from the first color, other pH indicator substances may respond to pH changes by undergoing a change from being colorless to having a specific color. Such pH indicators are well known in the art. For example, a solution of the indicator phenolphtalein changes it's appearance from colorless to pink or red at pH values of approximately 8.5 or higher. While phenolphtalein may not be usable in the gum or pH sensitive members of the present invention due to its laxative properties, other non-toxic or edible naturally obtained or synthetic pH indicators, exhibiting a transition from colorless to a specific color in the relevant range of pH 4.0-7.0 may be used in the gum or pH sensitive members of the present invention.

In accordance with another embodiment of the present invention, a dental gum kit is provided. The gum kit includes one or more pieces of chewing gum and one or more pH sensitive members for testing the oral fluids' or salivary pH before during or after the chewing of the dental gum.

The chewing gum piece or pieces included in the gum kit may be any suitable gum pieces. Preferably, the gum should be of type containing natural sweeteners which are not well metabolized by oral bacteria such as xylitol, sorbitol, maltitol syrup or the like, or artificial sweeteners such as aspartame, saccharine, cyclamate or the like, such that the sweetening substances in the gum minimally contribute to oral acidification. However, other suitable types of gum may also be used including dental gum compositions lacking sweeteners and/or containing other substances or compounds which are useful in oral hygiene, as long as none of the additives, alone or in combination with other additives, interferes with the pH indicating properties of the pH indicating substance(s) included in the gum base.

The pH sensitive members may be any pH sensitive members suitable for checking pH levels of saliva or oral fluids. For example, the pH sensitive member is the gum wrapper of the piece of gum or a portion thereof.

Reference is now made to FIG. 2 illustrating a wrapped chewing gum with pH indicator included in the wrapper, in accordance with a preferred embodiment of the present invention. The wrapped chewing gum 10 is wrapped in a wrapper 12 which incorporates a pH indicator with it (the gum itself is not shown here since it is wrapped in the wrapper 12).

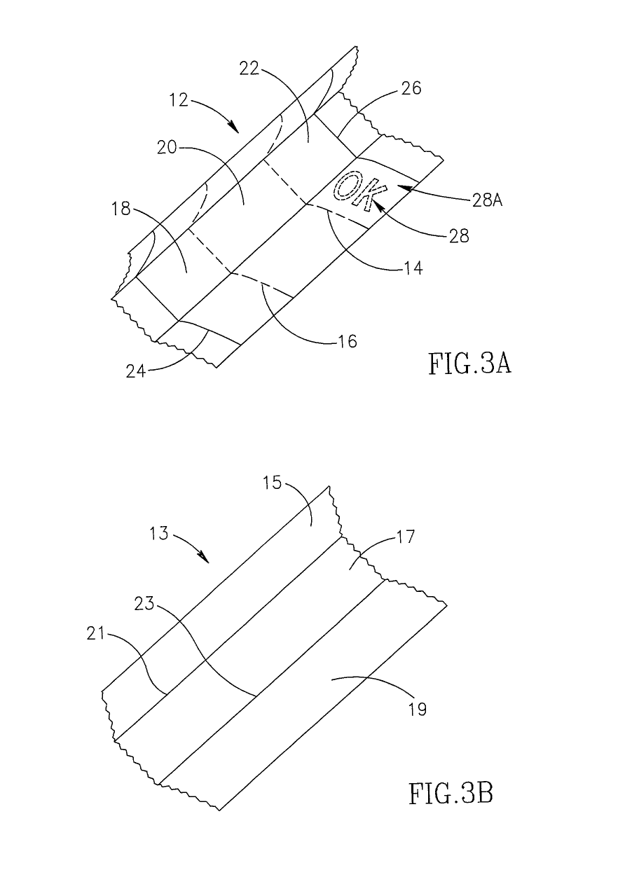

Reference is now made to FIG. 3A which is a schematic diagram illustrating the unfolded chewing gum wrapper 12 of FIG. 2 and the details of the pH indicator, in accordance with a preferred embodiment of the present invention. The wrapping sheet 1 contains a non-toxic pH indicator, which may be selected from the above mentioned substances (such as anthocyanin or other suitable substances or compounds). The consumer unfolds the wrapper and keeps it at hand. After chewing the gum for some time, the consumer may insert the wrapper, or a portion of the wrapper in his mouth and bring it in contact with his saliva or oral fluids. The pH indicator will display the appropriate color representing the current oral pH level.

Optionally, the wrapper is provided with one or more perforation lines dividing it into two or more portions. The wrapper 12 in FIG. 3A is provided with two perforation lines 14 and 16, which divide the wrapper into three portions 18, 20 and 22 (lines 24 and 26 are fold lines). Each of the wrapper portions 18, 20 and 22 contains a pH indicator substance. In accordance with one preferred embodiment of the present invention, all of the wrapper portions 18, 20 and 24 include the same pH indicator substance, such as an anthocyanin, a mixture of anthocyanins or the like, which may undergo a visibly detectable change color when the saliva or oral fluids sample put in contact with the wrapper portion is at a certain pH level, so that the consumer may examine his salivary or oral fluids pH levels at different times.

For example, if an anthocyanin containing substance is used, the wrapper 12 is impregnated with a non-toxic solution containing of anthocyanins such as the red cabbage extract catalog No. 979020 or the liquid Enocyanine solution catalog No. 979028, commercially available from Dr. Marcus Gmbh, Germany, and dried.

### EXAMPLE 3

A Sheet of home made red cabbage extract was prepared by boiling a few leaves of red cabbage in water, the resulting, red cabage extract was cooled and a piece of white absorbent paper was impregnated with the red cabbage extract and air dried to yield a light blue colored paper. This light blue colored paper was divided into three elongated strips.

The first strip of the light blue colored paper was wet with saliva by inserting half of it into the mouth immediately after drinking a glass of Coca Cola® soft drink, commercially available from the Coca Cola company. The color of the saliva wet portion of the paper strip changed it's color from light blue to light red.

The second strip of the light blue colored paper was wet with saliva by inserting half of it into the mouth immediately after chewing for a couple of minutes a piece of the Dental gum with baking soda, commercially available from Duane Reade, U.S.A.. The color of the saliva wet portion of the second paper strip changed it's color from light blue to green.

Generally, if the wrapper 12 or one of the portions 18, 20 and 22 thereof is dyed with a pH indicating dye and is put in contact with saliva or oral fluids obtained after a meal or a snack or a carbohydrate sweetened soft drink was consumed by the user, the acidic pH of approximately 4.0-4.5 results in the pH indicator having a first color. The first color indicates that the oral pH is under the critical pH level of pH 5.3 and that gum chewing should be started by the user (or continued if it had already been started). When the gum has been chewed for a sufficient amount of time, such that the salivary or oral fluid pH is larger than pH 5.3, contacting the wrapper 12 or a portion thereof with user saliva or oral fluids results in the pH indicator substance assuming a second color (for a pH of about 6.0).

Thus, when the wrapper 12 or one of the portions 18, 20 and 22 thereof develops the first color after being put in contact with saliva or oral fluids, this indicates to the user that the oral pH is still non-favorably acidic and that the chewing of the gum should be continued. When the wrapper 12 or portion thereof develops the second color after being put in contact with saliva or oral fluids, this indicates to the user that the oral pH is now favorably restored above the critical level of pH 5.3 and that the chewing of the gum may be stopped if desired.

While the wrapper 12 may be used for pH check as disclosed hereinabove without separating the different portions, the user may also tear the wrapper 12 along the perforated lines 14 and 16 to obtain three separated portions 18, 20 and 22 and may use one or more of the portions 18, 20 and 22 separately for repeatedly checking the pH of saliva or oral fluids. This may have the advantage of avoiding inadvertent wetting of the whole wrapper 12 by saliva or oral fluids, and of more conveniently enabling the repeating of the pH check by using a fresh portion of the wrapper 12 as the chewing of the gum proceeds.

It is noted that, while the wrapper 12 of FIGS. 2 and 3 has two perforated lines 14 and 16 allowing the separation of the wrapper 12 into three portions 18, 20 and 22 only, other preferred embodiments may be implemented (not shown) in which other different numbers of wrapper portions may be used, as long as the wrapper portions are of a manageable size which practically allows their use of pH checking.

It will be appreciated by those skilled in the art that the exact colors and hues of the pH indicator member at the different pH levels depend on the nature and composition of pH sensitive indicator compound or substance used. The exact type of color change and the pH range at which it happens may be varied by changing the composition of the pH indicator substance, or by blending various pH indicator substances, and may be adapted to happen at a particularly desired pH range which is regarded as clinically favorable for caries prevention, as long as the colors of the pH indicator substance(s) at the non-favorable and favorable oral fluid or salivary pH levels distinctly and visibly differ from each other.

In accordance with another preferred embodiment of the present invention, the wrapper 12 may be provided with different pH indicator substances, or different mixtures of pH indicator substances or compounds which display distinct different colors at different pH ranges. Thus, For example, the portion 18 of the wrapper 12, may be impregnated with a first pH sensitive substance or pH sensitive substance combination which undergoes a visibly detectable color change when exposed to saliva or oral fluids having a pH level within a first pH range, the portion 20 of the wrapper 12, may be impregnated with a second pH sensitive substance or pH sensitive substance combination which undergoes a visibly detectable color change when exposed to saliva or oral fluids having a pH level within a second pH range different than the first pH range, and the portion 22 of the wrapper 12, may be impregnated with a third pH sensitive substance or pH sensitive substance combination which undergoes a visibly detectable color change when exposed to saliva or oral fluids having a pH level within a third pH range, different than the first and second pH ranges.

The pH ranges within which distinct color changes occur may overlap or may not overlap. For example, in an overlapping pH ranges example, the portion 18 may change from red at pH 4 to violet at pH 5.3, the wrapper portion 20 may change from blue at pH 5.0 to green at pH 5.5, and the wrapper portion 22 20 may change from purple at pH 5.0 to violet at pH 6.5. In a non-overlapping range example, the portion 18 may change from red at pH 4 to violet at pH 5.0, the wrapper portion 20 may change from blue at pH 5.3 to green at pH 5.8, and the wrapper portion 22 20 may change from purple at pH 6.0 to violet at pH 7.0.

In the case of using various pH indicators undergoing visibly distinct color changes within different overlapping or non-overlapping pH ranges, it is suggested that the wrapper portions be marked or numbered, for example, by printing the numbers 1, 2, 3 on the wrapper portions, so that the numbers correspond to the order in which they are to be used. For example, the wrapper portion 18 may have the number 1 printed thereon (number not shown in FIG. 3A) and will be used first, just before, or at the commencing of the chewing, as a primary indication, when the oral pH level is expected to be low, and therefore it would contain a pH indicator substance or combination of substances which visibly changes color when exposed to saliva or oral fluids having a pH in the relatively low pH range (pH 4.0-5.0). The wrapper portion 20 may have the number 2 printed thereon (number not shown in FIG. 3A) and will be used second, about 5-10 minutes of chewing, and would contain a pH indicator substance or combination of substances which visibly changes color when exposed to saliva or oral fluids having a pH in the range of pH 5.0-5.5. Finally, the wrapper portion 22 may have the number 3 printed thereon (number not shown in FIG. 3A), and that would be used at about 20-30 minutes of chewing would contain a pH indicator substance or combination of substances which visibly changes color when exposed to saliva or oral fluids having a pH in the range of pH 5.5-6.0.

Once the pH indicator indicates that a favorable pH level has been reached the consumer may stop chewing the gum if he or she desires so.

Optionally, in accordance with another preferred embodiment of the present invention, the pH indication substance may be applied to a restricted area of the wrapper 12 such that the impregnated area is in the shape of a symbol or a mark 28 such as the letters OK, and be pre-designed to change its color after wetting with saliva or oral fluids such that the mark or symbol 28 clearly appears as distinguished from the background color of the wrapper 12 when the critical pH level of 5.3 pH disclosed hereinabove (or, optionally, any higher predetermined pH level) is reached.

For example, the wrapper area 28A may be dyed with a fast (preferably non-leaching) non-pH sensitive dye having a red color and the mark or symbol like area 28 is impregnated with a pH sensitive indicator dye having a red color which is similar in hue to the red color of the area 28A. When the areas 28 and 28A of the wrapper portion 22 are wet by saliva or oral fluids, if the salivary or oral fluid pH is not equal to or more basic than the critical pH of 5.3 (or any other desired pH level in the clinically favorable pH range), the color of the pH sensitive indicator dye does not change significantly and remains red. In this situation the symbol or mark 28 has the same color as the area 28A and is not clearly discernible on the red background of the area 28A.

If the salivary or oral fluid pH is equal to or more basic than the critical pH of 5.3 (or any other desired pH level in the clinically favorable pH range), the color of the pH sensitive indicator dye changes to violet-blue. In this situation, the violet-blue symbol or mark 28 clearly and distinctly stands out on the red background of the area 28A indicating that the desired clinically favorable pH level has been reached. The user may then stop the chewing of the gum if he desires to do so. It will be appreciated that the indicator substances and the background color may be modified and adapted such that the mark 28 will be clearly distinguished from the background color of the area 28A at other desired pH levels such as at pH 6, or any other desired pH level which is within the range of pH levels practically reachable by the saliva or oral fluids in the oral cavity.

It is noted that the marks or symbols, may also be shaped in any desired geometrical shapes, such as rectangles, circles, triangles and the like.

It is further noted that, while in the preferred embodiments disclosed hereinabove, the pH sensitive substances are impregnated within the actual paper of the wrapper 12 (FIGS. 2 and 3A), other preferred embodiments may be implemented in which the pH sensitive indicator substance or substances are first impregnated into separate flat pieces of a porous substance such as suitable absorbing paper or other flat sheets of a suitable porous absorbing material. Such pieces are dried and then attached to the wrapper by a suitable glue.

It is noted that while the chewing gum wrapper 12 of FIG. 3A is divided into different portions by the transverse perforated lines 14 and 16, other types of dividing a gum wrapper into a plurality of separate portions may be used.

Reference is now made to FIG. 3B which is a schematic diagram illustrating a gum wrapper which may be longitudinally separated into a plurality of pH sensitive members. The wrapper 13 includes three folds 15, 17 and 19 which may be folded when a piece of gum (not shown) is wrapped by the wrapper 13. The wrapper 13 may be separated into three separate portions by tearing it along the fold lines 21 and 23 or along perforated lines (not shown) passing along the fold lines 21 and 23. Each of the resulting separate strip like portions 15, 17 and 19 includes a pH sensitive substance or substances incorporated therein or within one or more discrete areas or patches therealong, as disclosed in detail for the wrapper 12 hereinabove. The number of pH sensitive wrapper portion may be increased by adding additional perforated lines (not shown) passing along the length of the wrapper 13, preferably but not necessarily in parallel to the fold lines 21 and 23.

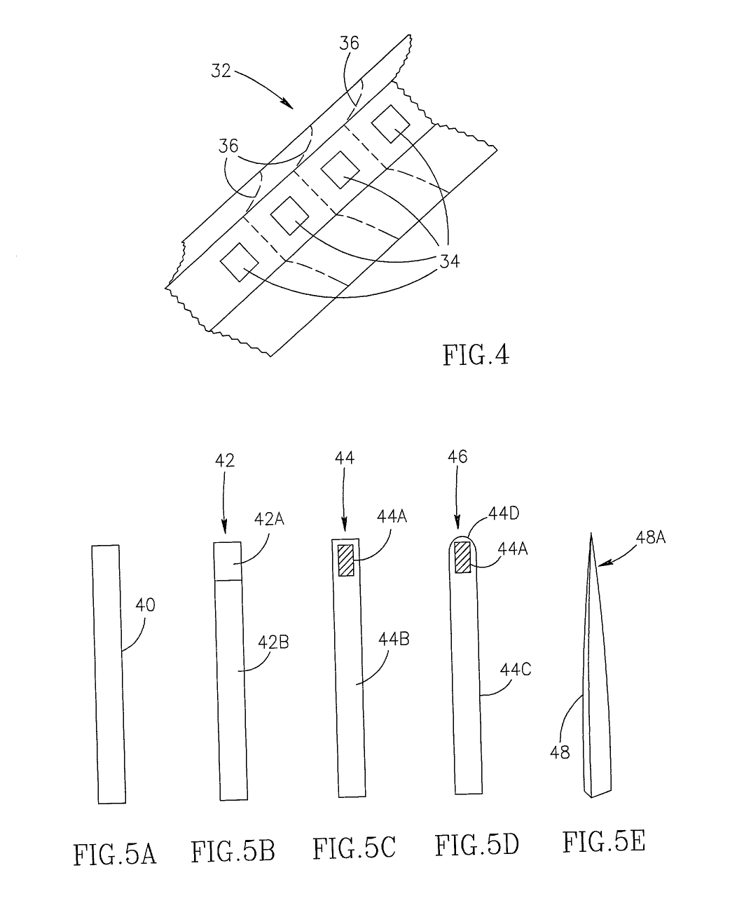

Reference is now made to FIG. 4 which is a schematic diagram of a chewing gum wrapper having a plurality of portions of paper impregnated with a pH sensitive indicator substance attached thereto, in accordance with a preferred embodiment of the present invention. The wrapper 32 has four pieces 34 attached thereto. The pieces 34 are made of suitable absorbent paper or another suitable type of flat absorbent material impregnated with a solution containing a pH indicator substance such as the anthocyanin or the red cabbage extract disclosed hereinabove and dried. The dry pieces 34 are attached to the wrapper 32 by any suitable glue which does not interfere with the pH determination or by any other suitable attachment method.

In using the wrapper 32, the user may selectively wet one of the pieces 34 for checking the pH as disclosed in detail hereinabove, or may also tear the wrapper 32 along the (optional) perforated lines 36 to obtain separate wrapper portions each having one of the pieces 34 attached thereto. Similar to the wrapper 12 of FIGS. 2 and 3A, all the pieces 34 may be impregnated with the same pH indicator substance or substance combination, and may be used for repeated pH checks before, during and after the chewing of the chewing gum.

Additionally, in accordance with another preferred embodiment of the invention, the indicator substance(s) may be included in restricted areas of the gum wrapper (not shown) by selectively impregnating the indicator substance(s) only in those restricted areas of the chewing gum wrapper (not shown). For example, this may be accomplished by stamping the wrapper with a stamp (not shown) having a predetermined shape and impregnated with the indicator substance(s), or by any other suitable application methods known in the art such as printing methods, stamping methods or the like. In contrast to impregnation methods in which the whole wrapper is impregnated with the same indicator substance, such stamping or printing methods may be advantageously applied for producing gum wrappers having a plurality of portions each incorporating different pH indicator compositions or substances as disclosed in detail hereinabove.

It is noted that while the gum wrapper may be used as the pH sensitive member in the dental gum kits of the present invention as disclosed in detail hereinabove, other forms of pH sensitive members may also be used.

Reference is now made to FIGS. 5A-5E which are schematic diagrams illustrating various forms of pH sensitive members, usable in dental gum kits for checking the pH level of saliva or oral fluids, in accordance with additional preferred embodiments of the present invention.

FIG. 5A illustrates a pH sensitive member 40. The pH sensitive member 40 is an elongated flat strip of absorbent material such as paper or cardboard or any other suitable porous or absorbent non-toxic material. The entire pH sensitive member 40 is impregnated with a suitable solution of a pH indicator substance, such as any of the non-toxic pH indicator substances disclosed in detail hereinabove. For example, the red cabbage extract catalog No. 979020 or the liquid Enocyanine solution catalog No. 979028, commercially available from Dr. Marcus Gmbh, Germany may be used, or any other suitable non-toxic, synthetic or natural indicator substance or substance mixture. After impregnation, the pH sensitive member 40 is dried. One or preferably a few pH sensitive members 40 may then be packed for use with the gum kit (not shown). In use, the pH sensitive member 40 may be wet with saliva or oral fluids by inserting it into the mouth and then taking it out of the mouth and observing the color which develops at the part of the pH sensitive member 40 which was wet by saliva or oral fluids.

An advantage of the pH sensitive member 40 is that, since it's entire length is impregnated with the pH indicator substance, any portion thereof may be used for pH checking.

FIG. 5B illustrates a pH sensitive member 42. The pH sensitive member 42 is similar to the pH sensitive member 40 of FIG. 5A, except that only a portion thereof 42A is impregnated with the solution of pH indicator substance and dried. The remaining portion 42B of the pH sensitive member 42 does not incorporate the pH indicator substance. In use, the user holds the part 42B and inserts the part 42A into the mouth for wetting it by saliva or oral fluids. Preferably, the paper or cardboard is white such that the portion 42A containing the pH indicator substance is colored by the pH indicator substance and is thus clearly distinguishable from the non-colored portion 42B. The advantage of the pH sensitive member 42 of FIG. 5B is that a smaller amount of pH indicator substance is need in making it compared to the pH sensitive member 40 of FIG. 5A.

FIG. 5C illustrates a pH sensitive member 44. The a pH sensitive member 44 includes an applicator 44B. The applicator 44B is an elongated, preferably flat strip, preferably made of a non porous, non-absorbing material such as plastic or the like. The pH sensitive member 44 further includes an indicator pad 44A. The indicator pad 44A is a piece of absorbent material such as, but not limited to, paper, cardboard, suitable wood or a suitable absorbent polymer material. The indicator pad incorporates a pH indicator substance as disclosed hereinabove. The indicator pad 44A may be prepared by impregnating the piece the porous material disclosed hereinabove in a solution of a suitable pH indicator substance and drying as disclosed hereinabove or by any other suitable method. The pH indicator pad 44A is attached to the applicator 44B as illustrated in FIG. 5C by gluing with a suitable non-toxic glue or by any other suitable attaching method known in the art. An advantage of the pH sensitive member 44 is that since the applicator 44B is made from a non absorbing material, it is easier to handle and will not bend due to softening caused by absorbing of excess saliva or oral fluids during or after insertion into the mouth.

FIG. 5D illustrates a pH sensitive member 46. The pH sensitive member 46 includes an applicator 44C and the pH indicator pad 44A. The pH sensitive member 46 of FIG. 5D differs from the pH sensitive member 44 of FIG. 5C in that the applicator 44C has a rounded end 44D. The rounded end 44D has the advantage of not having sharp corners which may be more convenient in use.

FIG. 5E illustrates a tooth-pick like pH sensitive member 48. The member 48 is shaped like a tooth-pick and tapers towards it's end 48A. Preferably, the pH sensitive member 48 is made of wood and may be shaped in any suitable shape such as, but not limited to, the shape of the dental tooth-picks commercially available under the trademark STIM-U-DENT® plaque removers, from Johnson and Johnson Consumer products Inc., U.S.A. The entire pH sensitive member 48 may incorporate a pH indicator substance or substances, such as any of the pH indicator substances disclosed hereinabove, or other suitable non-toxic pH indicators known in the art. Alternatively, only part of the pH sensitive member 48, such as the end 48A may incorporate the pH indicator substance. The pH indicator substance(s) may be applied to the pH sensitive member 48 or to any selected portion thereof, by any suitable method such as the impregnation and drying method disclosed hereinabove or my any other suitable method. Preferably, the wood or other material from which the pH sensitive member 48 is made is white or has a light color so as not to mask the color changes induced in the pH indicator substance(s) by the saliva or oral fluids. This may be achieved by simply selecting a white wood or a lightly colored wood for making the pH sensitive member 48, or by whitening or bleaching the wood or other material from which the pH sensitive member 48 is made prior to the application or incorporation of the pH indicator substance therein.

In use, the pH sensitive member 48 is used in a manner similar to the other pH sensitive member disclosed hereinabove and illustrated in FIGS. 5A-5D. However, while the pH sensitive member 48 may be used by contacting it or a portion thereof with saliva or oral fluids as disclosed hereinabove, it has an additional advantage that it may more easily inserted in the space between teeth and may thus possibly more accurately indicate the pH levels in the regions between the teeth which may be more difficult to access using the other different pH sensitive members 40, 42, 44 and 46 disclosed hereinabove. An additional advantage of pH sensitive member 48 of FIG. 5E is that it may also be used for the purpose of tooth picking helping to remove food remnants stuck between the teeth after a meal and may also be useful for removing dental plaque.

FIG. 5F illustrates a pH sensitive member 43. The pH sensitive member 43 is similar to the pH sensitive member 42 of FIG. 5B, except that it includes three portions 43A, 43B and 43C, each including a different pH indicator substance or a different pH indicating substance combination. The pH indicator substance or pH indicating substance combination included in each of the portions 43A, 43B and 43C may be adapted to develop a distinct color or hue when wet by oral fluids or saliva having a pH within a specific pH range. For example, The portion 43A may develop a red color at pH 4, the portion 43B may develop a purple color at pH 5 and the portion 43C may develop a blue color at pH 6. Thus, when the pH sensitive member 43 is used for pH check the user places the pH sensitive member 43 in his mouth to wet all the portions 43A, 43B and 43C with saliva or oral fluids. The resulting specific colors of each of the portions 43A, 43B and 43C may then be compared to a plurality color combinations on a suitable reference color scale (not shown) which is provided to the user as part of the dental gum kit.

Reference is now made to FIG. 5G which is a schematic diagram illustrating a reference color scale adapted for use with the pH sensitive member of FIG. 5F. The reference color scale 47 may be a paper or cardboard card or may be made from any other suitable material. The reference color scale 47 includes color marks 45A, 45B and 45C and an interpreting mark 45D associated with the color marks 45A, 45B and 45C. The interpreting mark 45D is printed text including the words “keep chewing”. The color marks 45A, 45B and 45C are areas of color printed on or otherwise applied to the surface of the reference color scale 47. The colors of the color marks 45A, 45B and 45C approximately represent the colors which are developed by the indicator substance(s) in the portions 43A, 43B and 43C, respectively, of the pH sensitive member 43 of FIG. 5F when the portions 43A, 43B and 43C are wet by saliva or oral fluids having a first selected pH level. For example, the colors of the color marks 45A, 45B and 45C may approximately represent the colors which are developed by the indicator substance(s) in the portions 43A, 43B and 43C, respectively, of the pH sensitive member 43 of FIG. 5F when the portions 43A, 43B and 43C are wet by saliva or oral fluids having a pH level of 5.3.

The reference color scale 47 also includes color marks 49A, 49B and 49C and an interpreting mark 49D associated with the color marks 49A, 49B and 49C. The interpreting mark 49D is printed text including the word “O.K.”. The color marks 49A, 49B and 49C are areas of color printed on or otherwise applied to the surface of the reference color scale 47. The colors of the color marks 49A, 49B and 49C approximately represent the colors which are developed by the indicator substance(s) in the portions 43A, 43B and 43C, respectively, of the pH sensitive member 43 of FIG. 5F when the portions 43A, 43B and 43C are wet by saliva or oral fluids having a second selected pH level. For example, the colors of the color marks 49A, 49B and 49C may approximately represent the colors which are developed by the indicator substance(s) in the portions 43A, 43B and 43C, respectively, of the pH sensitive member 43 of FIG. 5F when the portions 43A, 43B and 43C are wet by saliva or oral fluids having a pH level of 6.

In use, the user wets the portions 43A, 43B and 43C of the pH sensitive member 43 of FIG. 5F with saliva or oral fluids and compares the colors developed on the portions 43A, 43B and 43C with the colors of the color marks on the color scale 47. If the colors of the portions 43A, 43B and 43C resemble the colors of the color marks 45A, 45B and 45C of FIG. 5G, respectively, the user may interpret the check result in accordance with the interpreting mark 45D as a recommendation to keep chewing the gum. If the colors of the portions 43A, 43B and 43C resemble the colors of the color marks 49A, 49B and 49C of FIG. 5G, respectively, the user may interpret the check result in accordance with the interpreting mark 49D as indicating that the chewing the gum has restored the oral pH to a favorable pH (pH 6 in the particular, non-limiting example of the reference color scale 47), and may stop chewing the gum.

It is noted that the pH sensitive members 44, 46 of FIGS. 5C and 5D, respectively may also be modified to include a plurality of indicator pads (not shown) each including a different pH indicator substance or pH indicator substance combinations. Moreover, while the preferred embodiment illustrated in FIG. 5F includes three portions each having a different indicator substance or substances, other preferred embodiments of the pH sensitive may include different numbers of portions or indicator pads such as 2 or 4 portions or any other selected number of portions or indicator pads which is practically usable.

It is further noted that, care should be taken to avoid using materials in the construction of the pH sensitive members which may actively chemically interact with the pH indicator substance or interfere in an undesired manner with the saliva or oral fluid induced color changes in the pH indicator substance. For example, if the wood is pretreated with bleaching materials such as, for example chlorates, chlorine or peroxides, care must be taken to wash out or remove any traces of these materials from the wood since many indicator dyes may be irreversibly destroyed by bleaching or oxidizing materials.

It will be appreciated by those skilled in the art that the dimensions and shapes of the various components of the pH sensitive members 40, 42, 44, 46, 48 and 43 of FIGS. 5A, 5B. 5C, 5D, 5E and 5F, respectively, may be modified and that many shapes and dimensions may be used which are all included within the scope and spirit of the present invention.

It is noted that, the exact tints and hues of the colors at different pH levels of indicator substances such as the anthocyanin mixtures obtained from plant extracts such as red cabbage extract or the like, may exhibit some variations due to, inter alia, different extraction procedures, the nature and state of the vegetable materials used for preparing the pH indicator substance, seasonal changes and other factors. It may therefore be desired to provide the user of the present invention with a reference color scale. The reference color scale may be used to assist the user in interpreting the color of the chewing gum or of the pH sensitive members of the various preferred embodiments of dental gum kits of the present invention such as the wrappers 12 or 32 or portions thereof after being wet by the user's saliva or oral fluids.

The preparation and use of pH reference color scales is well known in the art and is not disclosed in detail. Typically, the reference color scale is a scale which provides samples of colors similar or identical to some of the colors which the pH sensitive substance or compound acquires at different selected reference pH levels. Such reference color scales are useful in assisting proper interpretation of the color changes occurring in pH indicators, particularly in cases where the colors gradually vary with pH change or when the differences in the indicator colors at different pH levels are subtle.

In accordance with one preferred embodiment of the present invention, the reference color scale (not shown) may be a separate reference color scale printed or otherwise applied to a piece of paper or any other suitable material. The reference color scale is included in the dental gum kit and may be used for assisting the user to interpret the color changes of the chewing gum during or after chewing the gum or to interpret the color changes of a pH sensitive member, such as but not limited to the pH sensitive members 40, 42, 44, 46 and 48 of FIGS. 5A, 5B, 5C, 5D and 5E, respectively, after it has been wet by saliva or oral fluids.

In accordance with another preferred embodiment of the present invention, the reference color scale (not shown) may be a reference color scale printed or otherwise applied to a gum wrapper such as, but not limited to the gum wrappers 12 or 32 disclosed hereinabove, or to the wrapper of the entire gum kit as disclosed hereinafter.

In accordance with yet another preferred embodiment of the present invention, the reference color scale (not shown) may be a reference color scale printed or otherwise applied onto or attached to a container (not shown) or chewing gum dispenser (not shown) which includes the gum kit.

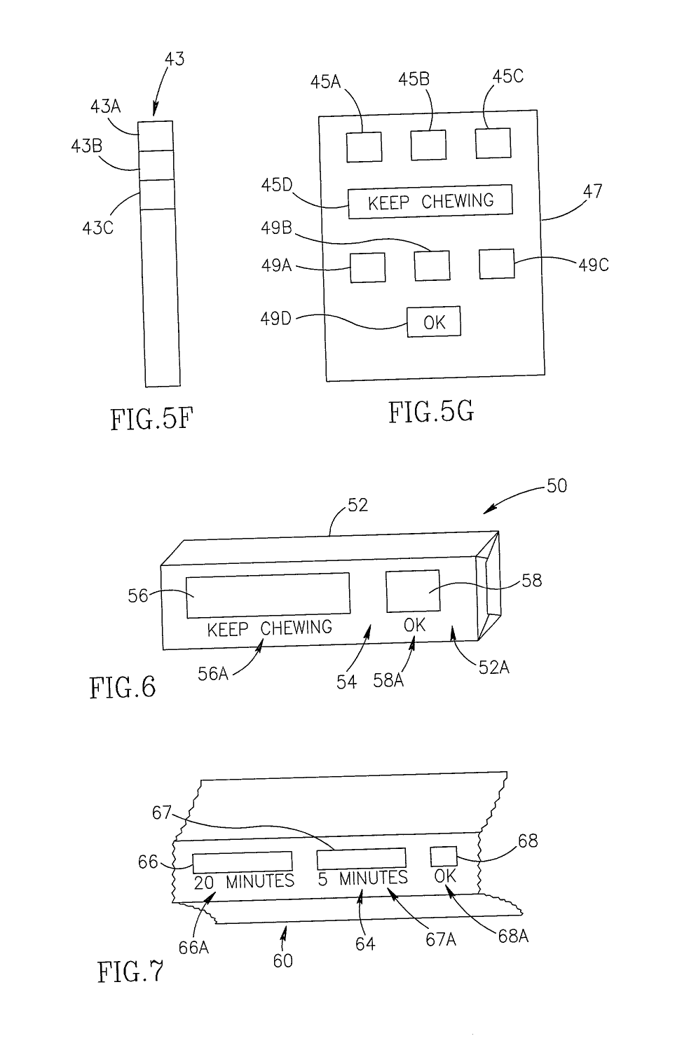

Reference is now made to FIG. 6 which is a schematic isometric view of a gum kit including a color reference scale positioned on the wrapper of the kit.

The kit 50 includes a one or more gum pieces (not shown), preferably of the type including pH indicator substance(s) incorporated therewithin as disclosed in detail hereinabove. The kit 50 further includes a kit wrapper 52 which is wrapped around the chewing gum pieces (not shown) included in the kit 50. The kit 50 further includes a reference color scale 54. The reference color scale 54 is printed on, or otherwise applied to the surface 52A of the kit wrapper 52. For example, the reference color scale 54 may be directly printed on the surface 52A or may be printed on another sheet of material (not shown) such as paper, plastic or the like which attached by glue to the surface 52A. The reference color scale 54 includes two reference color marks 56 and 58 which are printed on or otherwise applied or attached to the surface 52A. It is noted that the reference color marks 56 and 58 may actually comprise a color layer printed directly on the surface 52A or may also comprise actual pieces of colored material (not shown) attached or glued to the surface 52A. The reference color scale 54 may also include interpreting marks 56A and 58A associated with the reference color marks 56 and 58, respectively. The interpreting marks 56A and 58A of FIG. 6 are text marks printed or otherwise applied or attached besides or within (not shown in FIG. 6) the reference color marks 56 and 58.

In the kit wrapper 52 of FIG. 6, the interpreting mark 56A comprises the text “keep chewing” printed under the reference color mark 56. If after chewing one of the gum pieces included in the kit 50, the user observes that the color of the chewing gum piece is similar to the color of the reference color mark 56, the user may infer that the pH of the saliva or oral fluids is such that it is recommended to keep chewing the gum longer in order to raise the pH of the saliva or oral fluids to more favorable pH levels. The interpreting mark 58A comprises the text “O.K” printed under the reference color mark 58. If after chewing one of the gum pieces included in the kit 50, the user observes that the color of the chewing gum piece is similar to the color of the reference color mark 58, the user may infer that the pH level of the saliva or oral fluids is now restored to a favorable pH level and that he or she may stop chewing the gum if he or she so desires.

It is noted that other types of interpreting marks may be used such as differently worded instructions, the numbers representing the value of the actual pH levels corresponding with a gum color similar to the color of the color mark associated with the interpreting mark, or non-text like marks such as icons, pictures or other sign-like symbols which assist in the interpretation of the color of the chewing gum or provide instructions of how to proceed when the color of the gum is similar to the color of a selected one of the color marks 56 or 58.

It is further noted that while the kit 50 includes only one color scale 54, other embodiments may be implemented which contain more than one reference color scale. For example, another similar color scale may be printed on or otherwise applied to another surface (not shown) of the kit wrapper 52 such as the surface of the wrapper 52 on the side (not illustrated in FIG. 6) of the wrapper opposite to the surface 52A. Additionally, if the individual gum pieces (not shown) included in the kit 50 are wrapped by gum wrappers, each gum wrapper may also include a similar to, but not necessarily of the same dimensions or location to the reference color scale 54. This embodiment has the advantage that each individually wrapped piece of gum (not shown) becomes a fully functional self contained kit which includes the pH sensitive gum and a reference color scale for use with the gum. Thus, if the kit wrapper is lost or misplaced the checking may still be performed using a single wrapped gum piece.

It is yet noted that, while the kit 50 is disclosed as including pH sensitive gum pieces and one or more reference color scales, other implementations of the kit may be used. For example, a kit may include one or more gum pieces, one or more of the pH sensitive members disclosed hereinabove (either as part(s) of each gum wrapper or as separate pH sensitive members such as, but not limited to, the pH sensitive members 40, 42, 44, 46, and 48 disclosed hereinabove), and optionally a reference color scale (not shown) either as part of the container or wrapper of the whole kit or as an independent separate reference color scale packed within the kit or printed or attached to each individual gum wrapper. Thus, different combinations and permutations of the various gum kit components are generally possible.

Furthermore, when the gum wrapper includes the pH sensitive member such as in the case of the gum wrappers 12, 13 and 32, while generally it may be preferred to print the color scale on the side of the gum wrapper (not shown) which does not face the gum (not shown), it may become inconvenient to turn the wrapper or the portion of the wrapper to it's back side for the purpose of using the reference color scale. However, It may also be possible to print a color scale on a foldable portion on the side of the gum wrapper which does not face the gum while the pH indicator substance is placed or impregnated on the side facing the gum. After the gum wrapper or the portion thereof are used for checking the saliva or oral fluids as disclosed hereinabove. The wrapper portion bearing the color scale (not shown) may be folded over the other side so that both the reference color scale and the side or portion thereof with the pH indicator face the user, facilitating the use of the reference color scale.

It is still further noted that, while the reference color scale 54 includes only two reference color marks 56 and 58, other embodiments of the present invention may include more than three reference color marks.

Reference is now made to FIG. 7 which is a schematic isometric view of a gum wrapper including a reference color scale useful for indicating the recommended time of chewing a gum based on the results of a pH check of saliva or oral fluids, in accordance with a preferred embodiment of the present invention.

The gum wrapper 60 includes a reference color scale 64 printed thereupon or otherwise applied or attached thereto, as disclosed in detail hereinabove. The reference color scale 64 includes three reference color marks 66, 67 and 68. The reference color scale 64 also includes three interpreting marks 66A, 67B and 68A associated with the reference color marks 66, 67 and 68, respectively.

The interpreting mark 66A comprises the words “20 minutes” printed near the reference color mark 66. The interpreting mark 67A comprises the words “5 minutes” printed near the reference color mark 67. The interpreting mark 68A comprises the words “O.K.” printed near the reference color mark 68. In use, when the color of the chewing gum or the color of the pH sensitive member which was used to test the salivary or oral fluid pH resembles the color of the reference color mark 66, this may be interpreted by the user to mean that the user may need to chew the gum for approximately 20 minutes more to reach a favorable pH level (such as, but not limited to approximately pH 6).

When the color of the chewing gum or the color of the pH sensitive member which was used to test the salivary or oral fluids pH resembles the color of the reference color mark 67, this may be interpreted by the user to mean that the user may need to chew the gum for approximately 5 minutes more to reach the favorable pH level. When the color of the chewing gum or the color of the pH sensitive member which was used to test the salivary or oral fluids pH resembles the color of the reference color mark 68, this may be interpreted to mean that the salivary or oral fluids pH has reached a favorable pH level and that the user may stop chewing the gum if so desired. Thus, in accordance with the preferred embodiment, the interpreting marks 66A and 67A actually inform the user about the estimated times remaining for chewing the gum to achieve a desired salivary or oral fluids' pH level. These time values may be determined based on averages of times required for reaching certain selected pH values.

It is noted that while the reference color scale 64 of FIG. 7 includes three reference color marks (66, 67 and 68), the color scale 64 may be modified to include a different number of reference color marks (not shown). A higher number of reference color marks, such as, but not limited to five reference color marks may be advantageous by increasing the accuracy of the predicted remaining chewing time required for reaching a favorable salivary or oral fluids pH. However, the number of reference color marks may be restricted, inter alia, by the type and nature of the color changes available for the specific pH indicator substance(s) which are used, by practical manufacturing considerations, such as the available area on the wrapper and by other considerations such as avoiding making the checking procedure too complicated or time consuming.

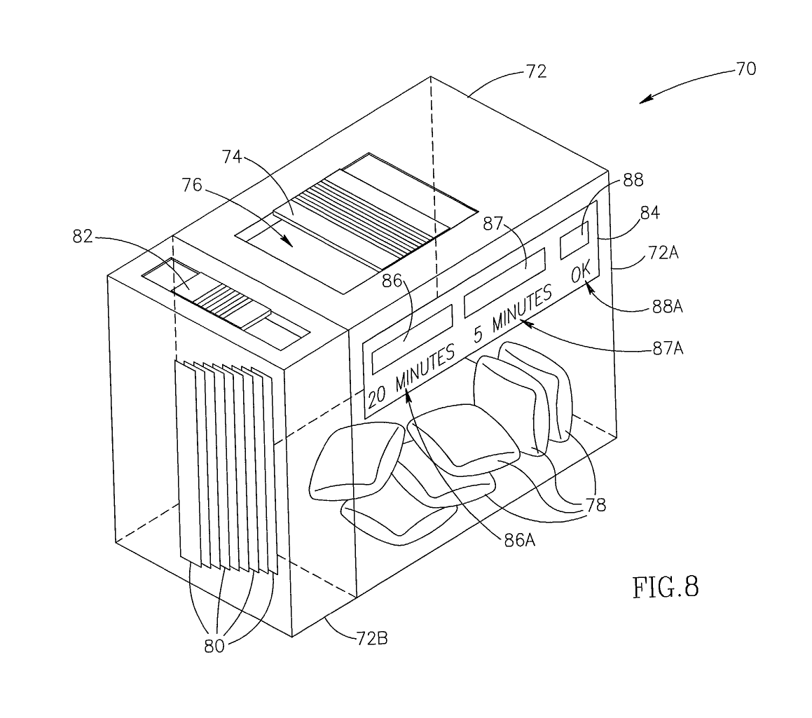

Reference is now made to FIG. 8 which is a schematic diagram of a dental gum kit, in accordance with another preferred embodiment of the present invention.

The dental kit 70 includes a dispenser 72. The dispenser 72 is a closeable container having two separate compartments, a gum dispensing compartment 72A and a pH sensitive member dispensing compartment 72B. The dispenser 72 may be made from any suitable material such as a transparent plastic or any other suitable material. The gum dispensing compartment 72A may include one or more gum pieces 78. The gum pieces are preferably of the types containing artificial sweeteners or may contain one or more of the sweeteners xylitol, sorbitol, maltitol syrup or other sweetening carbohydrates which are not metabolized or slowly metabolized by plaque bacteria and therefore minimally contribute to plaque acidification, as disclosed hereinabove, but other gum types may also be used.

The gum dispensing compartment 72A has a movable sliding cover 72B which may slide to open or close a dispensing opening 76 for dispensing one or more of the gum pieces 78. The dispensing compartment 72B is a compartment which includes a plurality of pH sensitive members 80. The compartment 72B has a sliding cover 82 which may be moved to open or close an opening (not shown) in the compartment 72B, for dispensing one or more of the pH sensitive members 80 stored in the compartment 72B. The pH sensitive members 80 may be any of the types of pH sensitive members, such as the pH sensitive members 40, 42, 44, 46 and 48 of FIGS. 5A, 5B, 5C, 5D, and 5E, respectively.

The gum kit 70 optionally includes a reference color scale 84 glued or otherwise attached to the dispenser 72. The color reference scale 84 includes three reference color marks 86, 87 and 88, and three interpreting marks 86A, 87A and 88A associated with the reference marks 86, 87 and 88, respectively.

The reference color marks 86, 87 and 88, and the interpreting marks 86A, 87A and 88A are implemented and used similarly to the corresponding color reference marks 66, 67 and 68 and interpreting marks 66A, 67A and 68A of FIG. 7.

In use, the user may dispense one or more gum pieces 78 from the dispenser 72 for chewing, and may dispense from the dispenser 72 one or more pH sensitive members 80 to perform oral fluids or salivary pH checks as disclosed in detail hereinabove. The user may elect to perform the pH checks at any time before, during and after the chewing of the gum. For example, the user may perform a check of oral pH after the consumption of a meal a snack or a beverage by dispensing a pH sensitive member 80 and using it as disclosed hereinabove. If the check reveals a pH level below the critical pH, the user may dispense a gum piece and start chewing it. The user may then dispense one or more additional pH sensitive members 80 and perform one or more additional pH checks within the duration of chewing the gum. The results of the latter pH check or checks may inform the user as to whether the gum e chewing should continue, and may optionally provide the user with an approximate estimate of the recommended duration of additional chewing, as disclosed in detail hereinabove.

It is noted that, the pH indicating chewing gum and the dental gum kits disclosed hereinabove may include a set of instructions explaining how to use the gum or the dental gum kit and how to interpret the color changes of the the pH indicating gum or of the pH sensitive members, with or without the assistance of a reference color scale. The instructions may be printed on the container or wrapper (not shown) containing the gum or the kit, or on the wrapper of each gum piece or may be included with the gum kit or gum container as a separate instruction sheet. Alternatively, if a reference color scale is provided with the gum piece, the entire dental gum kit or a container including gum pieces, the instructions for use may be printed on the reference color scale.

It is noted that the description of the embodiments and attached drawing figures set forth in this specification serves only for a better understanding of the invention, without limiting its scope as covered by the following claims.

It should also be clear that a person skilled in the art, after reading the present specification could make various modifications and variations to the described embodiments that are included within the scope and spirit of the present invention which is defined by the following claims.

What is claimed is:

1. A chewing gum for checking the pH of oral fluids, the chewing gum comprising:
a gum base; and
at least one pH sensitive substance dispersed in said gum base.

2. The chewing gum according to claim 1 wherein said at least one pH sensitive substance is capable of responding to the pH level of said oral fluids by undergoing a visibly detectable color change.

3. The chewing gum according to claim 1 further comprising at least one sweetening substance.

4. The chewing gum according to claim 1 wherein said at least one pH sensitive substance is adapted to responds to pH changes in the range of 4.0-6.0 by visibly changing its color.

5. The chewing gum according to claim 1 wherein said visibly detectable color change is selected from a visibly detectable change from a colorless to a colored form, a visibly detectable change from a colored form to a colorless form and a visibly detectable change from a first color to a second color.

6. The chewing gum according to claim 1 wherein said at least one pH sensitive substance comprises one or more anthocyanin dyes obtained from red cabbage, and wherein said visibly detectable color change comprises a change in the color of chewing gum from a red color at a pH of approximately 4 to a blue color at a pH of approximately 6.

7. The chewing gum according to claim 1 wherein said at least one pH sensitive substance is a non-toxic pH indicator dye.

8. The chewing gum according to claim 7 wherein said at least one pH sensitive substance is an anthocyanin pH indicator dye.

9. The chewing gum according to claim 7 wherein said at least one pH sensitive substance is an edible anthocyanin preparation obtained from red cabbage.

10. The chewing gum according to claim 1 further comprising at least one substance selected from an emulsifier, an antioxidant, a stabilizer and a flavoring substance.

11. The chewing gum according to claim 1 wherein said oral fluids are selected from saliva, a liquid introduced into the oral cavity, a liquid containing food particles dispersed therein, and any combination thereof.

12. A dental gum kit comprising:
at least one piece of chewing gum including a gum base and at least one pH sensitive substance dispersed in said gum base; and
a reference color scale for assisting a user of said gum kit to interpret the results of a pH check performed by chewing said at least one piece of chewing gum.

13. The dental gum kit according to claim 12 wherein said at least one pH sensitive substance is capable of responding to the pH level of said oral fluids by undergoing a visibly detectable color change.

14. A gum kit comprising:
at least one piece of chewing gum;
at least one pH sensitive member for checking the pH level of oral fluids.

15. The gum kit according to claim 14 wherein said at least one pH sensitive member is an elongated strip-like member having at least one portion comprising at least one pH sensitive substance capable of responding to the pH of oral fluids by undergoing a visibly detectable color change.

16. The gum kit according to claim 15 wherein said at least one pH sensitive member comprises an elongated absorbent member and wherein at least one portion of said elongated absorbent member is impregnated with said at least one pH sensitive substance.

17. The gum kit according to claim 16 wherein said at least one pH sensitive member is made of a material selected from wood, paper, and a porous absorbent synthetic polymer.

18. The gum kit according to claim 14 wherein said at least one pH sensitive member includes an applicator, and at least one piece of absorbent material impregnated with said at least one pH sensitive substance.

19. The gum kit according to claim 18 wherein said applicator is a plastic applicator and wherein said at least one piece of absorbent material is made of a material selected from wood, paper, and a porous absorbent synthetic polymer.

20. The gum kit according to claim 14 wherein said at least one piece of chewing gum is disposed within a gum wrapper, and wherein said at least one pH sensitive member comprises said gum wrapper or a portion thereof.

21. The gum kit according to claim 20 wherein said gum wrapper includes a reference color scale for assisting a user of said gum kit to interpret the results of an oral pH check performed using said gum wrapper or said portion thereof.

22. The gum kit according to claim 20 wherein said gum wrapper comprises a plurality of portions, each of said portions is usable for checking the pH of oral fluids.

23. The gum kit according to claim 22 wherein at least a first portion of said plurality of portions is adapted to undergo a visibly detectable color change within a first pH range and at least a second portion of said plurality of portions is adapted to undergo a visibly detectable color change within a second pH range.

24. The gum kit according to claim 23 wherein said first pH range and said second pH range at least partially overlap.

25. The gum kit according to claim 23 wherein said first pH range and said second pH range do not overlap.

26. The gum kit according to claim 22 wherein said plurality of portions are separable portions.

27. The gum kit according to claim 26 wherein said separable portions are separated from each other by tearing said wrapper along at least one tearing guide line.

28. The gum kit according to claim 27 wherein said at least one tearing guide line is selected from a fold line and a perforated guide line.

29. The gum kit according to claim 14 wherein said at least one pH sensitive member is a toothpick-like member having at least a portion thereof including said at least one pH sensitive substance.

30. The gum kit according to claim 29 wherein said toothpick-like member is further adapted to be used for performing oral hygiene operations selected from tooth picking and plaque removing.

31. The gum kit according to claim 14 wherein said oral fluids are selected from saliva, a liquid introduced into the oral cavity, a liquid containing food particles dispersed therein, and any combination thereof.

32. The gum kit according to claim 14 further including a reference color scale for assisting a user of said gum kit to interpret the results of a pH check performed using said at least one pH sensitive member.

33. The gum kit according to claim 32 wherein said gum kit further includes a container for holding said at least one piece of chewing gum and said at least one pH sensitive member, and wherein said reference color scale is disposed within said container.

34. The gum kit according to claim 33 wherein said container comprises a gum kit wrapper, and wherein said reference color scale is disposed within said gum kit wrapper or printed thereupon.

35. The gum kit according to claim 32 wherein said gum kit further includes a container for holding said at least one piece of chewing gum and said at least one pH sensitive member, and wherein said reference color scale is attached to or printed on said container.

36. A method for preparing chewing gum for checking the pH of oral fluids, the method comprising the steps of:
providing a chewable gum base; and
dispersing at least one pH sensitive substance in said gum base, said at least one pH sensitive substance is capable of responding to the pH level of said oral fluids by undergoing a visibly detectable color change.

37. The method according to claim 36 further including the step of dispersing at least one sweetening substance in said gum base.

38. The method according to claim 37 wherein said sweetening substance is selected from the group including sorbitol, xylitol, mannitol, maltitol syrup aspartame, sacharine, cyclamate and any combination thereof.

39. The method according to claim 36 wherein said at least one pH sensitive substance responds to pH changes in the range of 4.0-6.0 by undergoing a visibly detectable color change.

40. The method according to claim 36 wherein said visibly detectable color change is selected from a visibly detectable change from a colorless to a colored form, a visibly detectable change from a colored form to a colorless form and a visibly detectable change from a first color to a second color.

41. The method according to claim 36 wherein said at least one pH sensitive substance comprises one or more anthocyanin dyes obtained from red cabbage, and wherein said visibly detectable color change comprises a change in the color of said chewing gum from a red color at a pH of approximately 4 to a blue color at a pH of approximately 6.

42. The chewing gum according to claim 36 wherein said at least one pH sensitive substance is a non-toxic pH indicator dye.

43. The method according to claim 42 wherein said at least one pH sensitive substance is an anthocyanin pH indicator dye.

44. The method according to claim 42 wherein said at least one pH sensitive substance is an edible anthocyanin preparation obtained from red cabbage.

45. The method according to claim 36 further including the step of adding to said gum base at least one substance selected from a softening substance, a flavoring substance, an antioxidant substance and an emulsifier substance.

46. The method according to claim 36 wherein said oral fluids are selected from saliva, a liquid introduced into the oral cavity, a liquid containing food particles dispersed therein, and any combination thereof.

47. A method of preparing a gum kit for performing oral pH checks, the method comprising the steps of:
providing at least one piece of chewing gum; and
providing at least one pH sensitive member adapted for checking the pH level of oral fluids.

48. The method according to claim 47 wherein said at least one pH sensitive member is adapted for checking the pH level of oral fluids at a time selected from before the chewing of said chewing gum, during the chewing of said chewing gum and after the chewing of said chewing gum.

49. The method according to claim 47 wherein said oral fluids are selected from saliva, a liquid introduced into the oral cavity, a liquid containing food particles dispersed therein, and any combination thereof.

50. A method of preparing a gum kit for checking oral fluids pH level, the method comprising the steps of:
providing at least one piece of chewing gum; and
providing means for checking said oral fluids pH level.

51. The method according to claim 50 wherein said means for checking said oral pH level is adapted for checking said oral fluids pH level at a time selected from before the chewing of said chewing gum, during the chewing of said chewing gum and after the chewing of said chewing gum.

52. The method according to claim 50 wherein said means for checking said oral pH level comprises at least one pH sensitive member capable of responding to the pH of oral fluids by undergoing a visibly detectable color change.

53. The method according to claim 50 wherein said means for checking said oral pH level comprises at least one piece of pH sensitive gum capable of responding to the pH level of oral fluids by undergoing a visibly detectable color change.

54. The method according to claim 50 further including the step of providing a reference color scale for assisting a user of said chewing gum to interpret changes in the color of said means for checking said oral pH level, said changes in color resulting from chewing said gum.

55. The method according to claim 50 wherein said oral fluids are selected from saliva, a liquid introduced into the oral cavity, a liquid containing food particles dispersed therein, and any combination thereof.

56. A chewing gum wrapper comprising a wrapping sheet, at least a portion of said wrapping sheet includes at least one pH sensitive substance.

57. The chewing gum wrapper according to claim 56, wherein said at least one pH sensitive substance is sensitive to pH level changes, and is capable, after being wet by oral fluids, of responding to the pH level of said oral fluids by undergoing a visibly detectable color change.

58. The chewing gum wrapper according to claim 47 wherein said oral fluids are selected from saliva, a liquid introduced into the oral cavity, a liquid containing food particles dispersed therein, and any combination thereof.
